(19) United States
(12) Patent Application Publication
(10) Pub. No.: US 2021/0145309 A1
(43) Pub. Date: May 20, 2021

(54) Physiological Monitoring Device and Method

(71) Applicant: 9681345 Canada Inc., Montreal, Quebec (CA)

(72) Inventor: Oscar Javier Acosta Palacios, Montreal (CA)

(21) Appl. No.: 17/047,290

(22) PCT Filed: Apr. 15, 2019
(86) PCT No.: PCT/CA2019/050459

§ 371 Date: Oct. 13, 2020

Related U.S. Application Data
(60) Provisional application No. 62/657,065, filed on Apr. 13, 2018.

(51) Int. Cl. A61B 5/08; A61B 5/01; A61B 5/024; A61B 5/113; A61B 5/00

(52) U.S. Cl. CPC A61B 5/0816; A61B 5/01; A61B 5/02416; A61B 2562/0271; A61B 5/6831; A61B 5/6823; A61B 5/1135

(57) ABSTRACT

A physiological monitoring method and device, the device comprising: a fixation band adapted to be fastened around a user's torso; a monitoring assembly fastened to the fixation band, the monitoring assembly comprising: at least one deformable member adapted to be deformed in response to an expansion of the user's torso; at least one deformation sensor operatively connected to the at least one deformable member for measuring a deformation of the deformable member; a processing unit operatively connected to the at least one sensor for determining a physiological parameter value of the user based on the measured deformation.

### CROSS-REFERENCE TO RELATED APPLICATIONS

The present patent application claims the benefits of priority of US Provisional Patent Application No. U.S. 62/657,065, entitled “Physiological Monitoring Device and Method” and filed at the United States Patent and Trademark Office” on Apr. 13, 2018.

### FIELD OF THE INVENTION

The present invention generally relates to physiological monitoring devices and methods for monitoring at least one physiological parameter of a user.

### BACKGROUND OF THE INVENTION

Physiological monitoring devices come in various forms, such as wirelessly connected fitness bracelets or watches, and are adapted to enable users to monitor various physiological parameters such as their heart rate in addition to other data such as the total steps made in a day, the distance travelled, the active minutes or even the calories burnt during a certain period. Some devices may further comprise advanced functionalities such as GPS positioning and wireless synchronization with a wireless apparatus such as a smart phone or a computer.

However, these devices are often limited in the type of data collected during physical activities and do not provide capabilities for monitoring other data such as the breathing rate, the oxygen saturation and the body temperature.

There is therefore a need for an improved physiological monitoring device which may overcome at least one of the previously identified drawbacks.

### SUMMARY OF THE INVENTION

The aforesaid and other objectives of the present invention are realized by generally providing a novel physiological monitoring device and method.

In accordance with one aspect of the invention, there is provided a physiological monitoring device comprising a fixation band adapted to be fastened around a user's torso, a monitoring assembly fastened to the fixation band, the monitoring assembly comprising at least one deformable member adapted to be deformed in response to an expansion of the user's torso, at least one deformation sensor operatively connected to the at least one deformable member for measuring a deformation of the deformable member, and a processing unit operatively connected to the at least one sensor for determining a physiological parameter value of the user based on the measured deformation.

The physiological parameter value may further comprise a respiratory rate of the user. The deformable member may further comprise a deformable substrate plate. The deformable substrate plate may be made from polycarbonate.

In one embodiment, the deformable substrate plate has a thickness of about 2 mm.

In one embodiment, the deformable substrate plate is generally rectangular.

In one embodiment, the deformable substrate plate comprises four anchoring holes disposed at the four corners of the deformable substrate plate.

In one embodiment, each one of the at least one deformation sensor is disposed at the center of a corresponding one of the at least one deformable substrate plate.

In one embodiment, the deformation sensor is disposed and oriented on the corresponding deformable substrate plate so as to allow measurements of strain longitudinally along the fixation band and the monitoring assembly.

In one embodiment, each deformation sensor comprises a strain gauge.

In one embodiment, each strain gauge comprises a foil strain gauge.

In one embodiment, the monitoring assembly further comprises a heart rate sensor.

In one embodiment, the heart rate sensor comprises a green light emitting diode and a corresponding photoreceptor.

In one embodiment, the monitoring assembly further comprises an oxygen saturation sensor.

In one embodiment, the oxygen saturation sensor comprises an infrared light emitting diode and a corresponding photoreceptor.

In one embodiment, the monitoring assembly further comprises a body temperature sensor.

In one embodiment, the monitoring assembly further comprises a housing adapted to house the at least one deformable member, the at least one deformation sensor and the processing unit.

In one embodiment, the housing is overmolded over the at least one deformable member, the at least one deformation sensor and the processing unit.

In one embodiment, the monitoring assembly further comprises a battery operatively connected to the processing unit.

In one embodiment, the battery is rechargeable.

In one embodiment, the monitoring assembly further comprises a communication unit operatively connected to the processing unit for allowing data to be exchanged between the monitoring assembly and at least one external terminal.

In one embodiment, the at least one external terminal comprises at least one of a smart phone, a smart watch and a personal computer.

In one embodiment, the monitoring assembly further comprises an antenna operatively connected to the communication unit for enabling data to be transmitted wirelessly by the communication unit.

In one embodiment, the monitoring assembly further comprises a memory for storing data received by the processing unit from the at least one sensor.

In one embodiment, the fixation band comprises an outer surface adapted to be disposed away from the user's torso and an inner surface adapted to be disposed towards the user's torso, the inner surface being textured to improve friction between the fixation band and the user's skin.

In accordance with another aspect of the invention, there is also provided a method for measuring a respiratory rate of a user, the method comprising providing a monitoring assembly comprising at least one deformable member adapted to be deformed in response to an expansion of the user's torso and at least one deformation sensor operatively connected to the at least one deformable member for measuring a deformation of the deformable member, placing the monitoring assembly against a user's torso using a fixation band attached to the monitoring assembly and measuring a strain value generated by the at least one deformation sensor in response to a plurality of inhalations and exhalations of the user over a predetermined period of time.

In one embodiment, measuring a strain value may further comprise measuring an increase in strain value from the at least one deformation sensor, the increase being indicative of an inhalation by the user and measuring a decrease in strain value from the at least one deformation sensor, the decrease being indicative of an exhalation by the user.

The features of the present invention which are believed to be novel are set forth with particularity in the appended claims.

### BRIEF DESCRIPTION OF THE DRAWINGS

The above and other objects, features and advantages of the invention will become more readily apparent from the following description, reference being made to the accompanying drawings in which:

FIG. 1 is a schematic view of a physiological monitoring device installed on a torso of a user, in accordance with one embodiment;

FIG. 2 is a schematic top plan view of a monitoring assembly for the physiological monitoring device illustrated in FIG. 1;

FIG. 3 is a schematic side elevation view of a monitoring assembly for the physiological monitoring device illustrated in FIG. 1;

FIG. 4 is a schematic top perspective view of a breathing sensor assembly for the physiological monitoring device illustrated in FIG. 1;

FIG. 5 is a partial perspective view of an inner surface of a fixation band for the physiological monitoring device illustrated in FIG. 1;

FIG. 6 is a front elevation view of the inner surface of the fixation band illustrated in

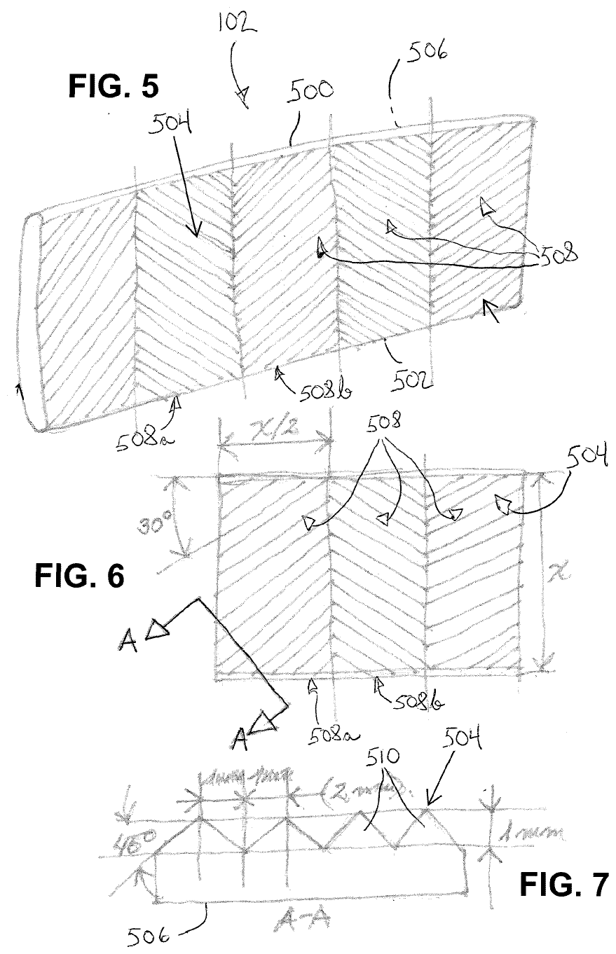

FIG. 5;

FIG. 7 is a cross-section view of the fixation band illustrated in FIG. 5, taken along line A-A;

FIG. 8 is a partial perspective view of an inner surface of a fixation band for a physiological monitoring device, in accordance with an alternative embodiment;

FIG. 9 is a front elevation view of the inner surface of the fixation band illustrated in FIG. 8;

FIG. 10 is a cross-section view of the fixation band illustrated in FIG. 8, taken along line A-A;

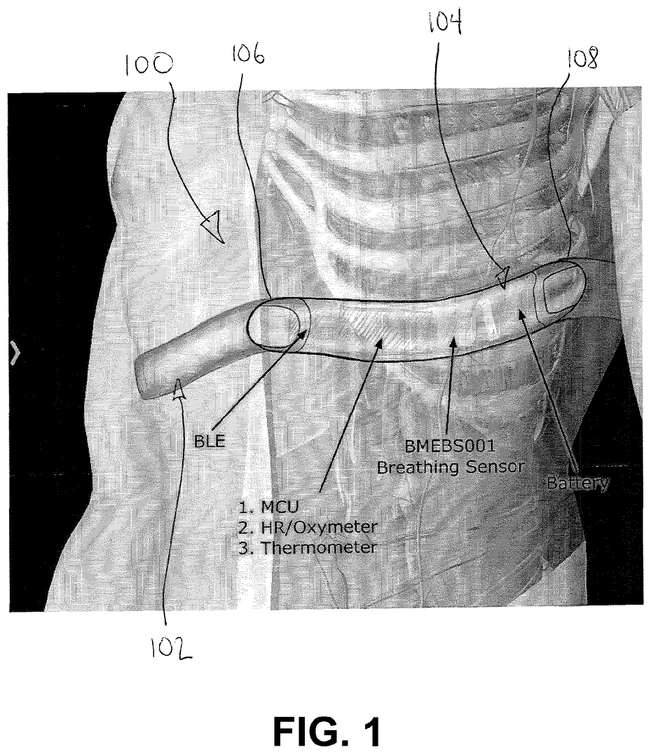

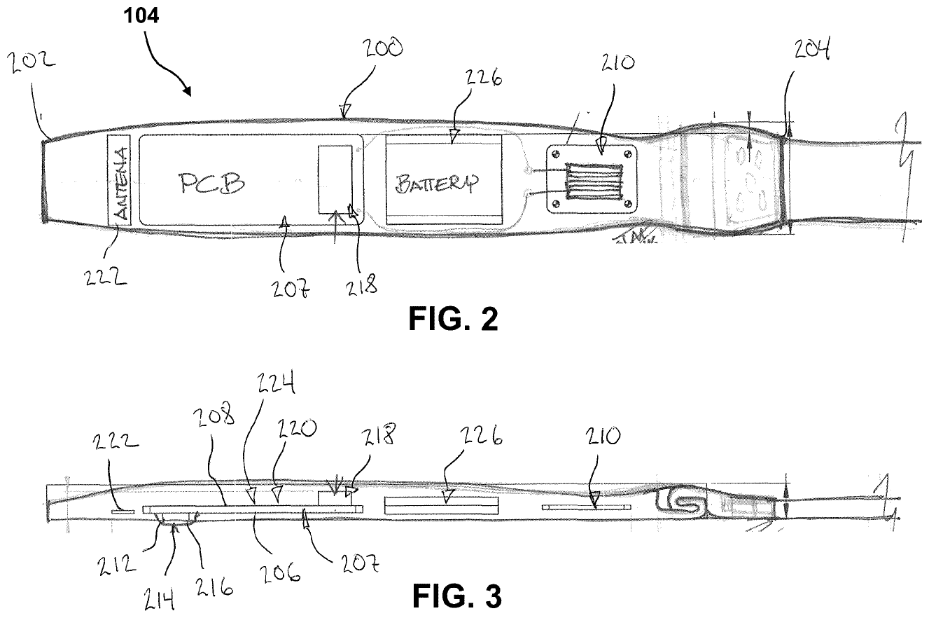

FIG. 11 is a schematic top perspective view of an attachment member for attaching the fixation band to the monitoring assembly of the physiological monitoring device illustrated in FIG. 1, in accordance with one embodiment;

FIG. 12 is a schematic top elevation view of the attachment member illustrated in FIG. 11, attached to the monitoring assembly;

FIG. 13 is a schematic side elevation view of the attachment member illustrated in FIG. 11, attached to the monitoring assembly;

FIG. 14 is a schematic top perspective view of an attachment member for attaching the fixation band to the monitoring assembly of the physiological monitoring device illustrated in FIG. 1, in accordance with an alternative embodiment;

FIG. 15 is a schematic top elevation view of the attachment member illustrated in FIG. 11, attached to the monitoring assembly;

FIG. 16 is a schematic side elevation view of the attachment member illustrated in FIG. 11, attached to the monitoring assembly;

FIG. 17 is a block diagram showing a monitoring assembly for the physiological monitoring device illustrated in FIG. 1;

FIG. 18 is line chart showing an example of a user's respiratory percentage volume as a function of time, measured using the device illustrated in FIG. 1; and

FIG. 19 is a schematic view of a physiological monitoring device installed on a torso of a user, in accordance with an alternative embodiment.

### DETAILED DESCRIPTION OF THE PREFERRED EMBODIMENT

A novel physiological monitoring device and method will be described hereinafter. Although the invention is described in terms of specific illustrative embodiment(s), it is to be understood that the embodiment(s) described herein are by way of example only and that the scope of the invention is not intended to be limited thereby.

Referring first to FIG. 1, there is shown a physiological monitoring device 100, in accordance with one embodiment. The physiological monitoring device 100 may be configured to monitor a wide variety of physiological parameters such as heart rate, body temperature, breathing rate and oxygen saturation of a user, especially during physical exercise.

The device 100 comprises a fixation band 102 adapted to be fastened around a user's torso and a monitoring assembly 104 fastened to the fixation band 102. Specifically, the fixation band 102 comprises a first end 106 and a second end 108 which are connected together by the monitoring assembly 104. The fixation band 102 is adapted to maintain the monitoring assembly 104 at a predetermined level on the user's torso. In the illustrated embodiment, the fixation band 102 is adapted to maintain the monitoring assembly 104 over the intercostal valley formed between the fifth and sixth true ribs on a user's torso. Alternatively, the fixation band 102 could be adapted to maintain the monitoring assembly 104 at a different location and/or height on the user's torso.

In the illustrated embodiment, the fixation band 102 is adapted to maintain the monitoring assembly 104 against the user's torso. Alternatively, the user could be wearing an upper body garment such as a shirt or an undershirt and the fixation band 102 could be adapted for maintaining the monitoring assembly 104 against the upper body garment and pressing the upper body garment against the user's torso.

Specifically, the fixation band 102 may be made from an elastomeric material such as silicone rubber or the like. It will be appreciated that silicone rubber is generally soft and flexible and thus would make a relatively comfortable fixation band. In one embodiment, the fixation band 102 is made using silicone rubber having a durometer rating of 60. Alternatively, the fixation band 102 could be made using a different material.

Now turning to FIGS. 2 and 3, the monitoring assembly 104 comprises a housing 200 for housing components of the monitoring assembly 104. In the illustrated embodiment, the housing 200 is made of an elastomeric stretchable, impact resistant material which is overmolded over the components of the monitoring assembly 104. The housing 200 has a first housing end 202 adapted to be attached to the first end 106 of the fixation band 102 and a second housing end 204 adapted to be attached to the second end 108 of the fixation band 102. In the illustrated embodiment, the housing 200 is elongated and thereby defines an extension of the fixation band 102, such that the housing 200 and the fixation band 102 together define a single, continuous loop around the user's torso.

In the illustrated embodiment, the housing 200 houses a controller 205 and a plurality of sensors operatively connected to the controller 205, each sensor being adapted for measuring at least one physiological parameter of the user. In the illustrated embodiment, the controller 202 comprises a printed circuit board or PCB 207 which is generally planar and has a bottom face 206 which is adapted to be disposed against the user's torso and a top face 208 opposite the bottom face 206. Still in this embodiment, the plurality of sensors comprise a breathing sensor assembly 210, a heart rate sensor 212, an oxygen saturation sensor 214 and a body temperature sensor 216, all operatively connected to the controller 205. Specifically, the heart rate sensor 212, the oxygen saturation sensor 214 and the body temperature sensor 216 may be mounted to the bottom face 206 of the PCB 207 to be in close proximity to the user's torso, as will be further explained below.

In the illustrated embodiment, the controller 205 comprises a processing unit 218 and a communication unit 220 operatively connected to the processing unit 218, both of which are mounted on the top face 208 of the PCB 207. In one embodiment, the controller 205 could further comprise an antenna 222 operatively connected to the communication unit 220 for enabling data to be transmitted wirelessly by the communication unit 220. In one embodiment, the controller 205 may further comprise a memory 224 operatively connected to the processing unit 218 for storing data received by the processing unit 218 from the sensors 210, 212, 214, 216. The processing unit 218 could comprise a microcontroller unit or MCU, the communication unit 220 could comprise a Bluetooth low energy (BLE) chip and the memory 224 could comprise a solid state memory card, for example. Alternatively, the processing unit 218, the communication unit 220 and the memory 224 could comprise different components.

In the illustrated embodiment, the controller 205 is further operatively connected to a portable energy source, such as a battery 226, which is adapted to provide power to the processing unit 218, the communication unit 220 and/or the sensors 210, 212, 214, 216. In the illustrated embodiment, the battery 226 is located next to the PCB 207 rather than being directly mounted to the PCB 207. Alternatively, the battery 226 could instead be mounted directly on the PCB 207. The controller 205 may further comprise a connection port which can be located on the battery 226 or on the PCB 207 for recharging the battery 226. For example, the battery 226 could comprise a 3.7 V ion lithium polymer 150 mAh rechargeable battery. Alternatively, the battery 226 could comprise a disposable battery and be removably mounted in the monitoring assembly 104. In yet another embodiment, the battery 226 could be adapted to be recharged wirelessly.

In one embodiment, the connection port may be a multi-purpose port which is further adapted for connecting the controller 205 to an external communication network, not shown, in order to allow data to be exchanged between the controller 205 and at least one external terminal such as a smart phone, a smart watch, a personal computer or the like. In this configuration, the controller 205 may be adapted to export data from the processing unit 218 and/or from the memory 224 to an external storage and/or to import data from an external source, for example to perform a firmware update. In this embodiment, the connection port may be accessible through a connection port access opening defined in the housing 200 which is sized and shaped accordingly.

In one embodiment, the controller 205 further comprises a power switch adapted to allow the controller 205 to be selectively activated and deactivated by the user. In this embodiment, the power switch could be accessible through a switch access opening defined in the housing 200 which is sized and shaped accordingly. It will be appreciated that deactivating the controller 205 and thereby turning off the device 100 when not in use contributes to preserving energy stored in the battery 226.

In one embodiment, the controller 205 could further be adapted to provide at least one indication, such as an indication that the controller 205 is powered, an indication that the sensors 210, 212, 214, 216 are measuring data, an indication of an energy level of the battery 226, or any other appropriate indication.

Specifically, the controller 205 could comprise at least one visual indicator operatively connected to the processing unit 218 for providing at least one visual indication. In one embodiment, the visual indicator could comprise an indicator light emitting diode or LED, and more specifically a tri-colour or RGB LED, mounted to the PCB 207 and adapted to be disposed away from the user to allow the user or another person standing away from the user to visualize the indicator LED and thereby receive indications from the indicator LED. The indicator LED could be adapted to provide these indications by one of being turned on, being turned on at a predetermined intensity, flashing, flashing at a predetermined rate, displaying a predetermined colour, or any combination of the above. In this embodiment, the housing 200 could be generally opaque or translucent but comprise a transparent viewing portion aligned with the indicator LED and adapted to be disposed away from the user's torso to allow the user and/or a person standing away from the user to view the indicator LED. Alternatively, the housing 200 could instead comprise a viewing opening defined in the housing 200 in alignment with the indicator LED. In yet another embodiment, the entire housing 200 could be translucent or transparent to thereby allow the indicator LED to be viewed.

In one embodiment, the controller 205 could further comprise a haptic vibration actuator which could be mounted on the bottom face 206 of the PCB 207 and which could be operatively connected to the processing unit 214 to provide a tactile indication according to a predetermined rhythm instead of a visual indication. In a further embodiment, the controller 205 could comprise both an indicator LED and a haptic vibration actuator to provide both visual and tactile indications.

In one embodiment, the heart rate sensor 212 is of the optical type and comprises a light source such as a green light emitting diode or green LED, and a photosensor. It will be appreciated that this type of sensor is adapted to be placed against the skin of the user. Alternatively, the heart rate sensor 212 could comprise any type of heart rate sensor known to a skilled person.

In one embodiment, the oxygen saturation sensor 214 is also of the optical type and comprises an infrared light source such as an infrared light emitting diode or infrared LED, and a photosensor adapted to measure an oxygen saturation rate of the user using known pulse oximetry methods. It will be appreciated that this type of sensor is also adapted to be placed against the skin of the user. Alternatively, the oxygen saturation sensor 214 could comprise any type of oxygen saturation sensor known to a skilled person.

In the above embodiment, the housing 200 could be generally opaque or translucent but comprise a transparent operative portion disposed towards the user's torso aligned with the green LED and/or the infrared LED to allow light from the green LED and the infrared LED to reach the user's skin. Alternatively, the housing 200 could instead comprise two distinct transparent operative portions, each one being in alignment with a respective one of the green LED and the infrared LED. In another embodiment, the housing 200 could instead comprise an operative opening defined in the housing 200 aligned with the green LED and/or the infrared LED instead. In yet another embodiment, the entire housing 200 could be translucent or transparent to thereby allow light from the green LED and the infrared LED to reach the user's skin through the housing 200.

In one embodiment, the body temperature sensor 216 comprises a temperature sensor known by a skilled person such as an integrated circuit (IC) temperature sensor, a thermistor, a resistance temperature detector (RTD), a thermocouple, an infrared (IR) temperature sensor or any other type of temperature sensor which a skilled person would deem to be appropriate for use in the device 100. It will be appreciated that the temperature sensor may also be adapted to be placed against the skin of the user.

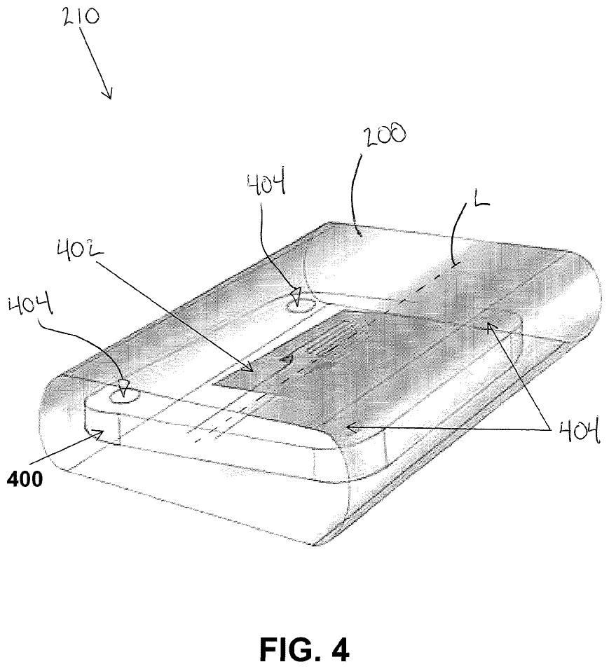

Referring now to FIG. 4, the breathing sensor assembly 210 comprises a deformation sensor which comprises a substrate plate 400 and a strain gauge 402 mounted on the substrate plate 400 to measure strain on the substrate plate 400. In one embodiment, the strain gauge 402 comprises a foil strain gauge secured on the substrate plate 400 using securing techniques known to a skilled person such as gluing with an appropriate adhesive or the like.

Specifically, the strain gauge 402 could comprise a HBM No. 6 SG Series Y or G—One Grid 6×13 mm-350Ω Strain Gauge. Alternatively, the strain gauge 402 could comprise another type of strain gauge such as a piezoresistor or any other type of strain gauge which a skilled person would consider to be appropriate.

In the illustrated embodiment, the substrate plate 400 is generally rectangular and comprises four anchoring holes 404 disposed generally at the four corners of the substrate plate 400. Each anchoring hole 404 is adapted for receiving a fastener to enable the substrate plate 400 to be securely affixed inside the housing 200.

In one embodiment, the substrate plate 400 has a thickness of about 2 mm and is made of polycarbonate. Alternatively, the substrate plate 400 could have another configuration and/or could instead be made of another material which is a skilled person would consider to be appropriate.

Still in the illustrated embodiment, the strain gauge 402 is generally disposed at the centre of the substrate plate 400. The strain gauge 402 is disposed and oriented on the substrate plate 400 so as to allow measurements of strain on the substrate plate 400, which corresponds to strain generated longitudinally along the fixation band 102 and the monitoring assembly 104. It will be understood that this strain is created by the expansion of the user's torso during inhalation. Specifically, an increase in measured strain value is indicative of an inhalation by the user, and a decrease in measured strain value is indicative of an exhalation by the user, as will be explained further below. In one embodiment, an excitation voltage is provided to the strain gauge 402 by the battery 226 and the measured strain value comprises a voltage value from the strain gauge 402 corresponding to strain on the substrate plate 400.

In the illustrated embodiment, the strain gauge 402 is generally elongated and defines a central longitudinal axis L. Still in the illustrated embodiment, the strain gauge 402 is oriented on the substrate plate 400 such that the gauge's longitudinal axis L is generally parallel to the fixation band's longitudinal axis. In this configuration, the gauge's longitudinal axis L is generally horizontal when the device 100 is worn by the user. Alternatively, the strain gauge 402 may be oriented on the substrate plate 400 such that the gauge's longitudinal axis L is generally perpendicular to the fixation band's longitudinal axis. In this configuration, the gauge's longitudinal axis L would be generally vertical when the device 100 is worn by the user.

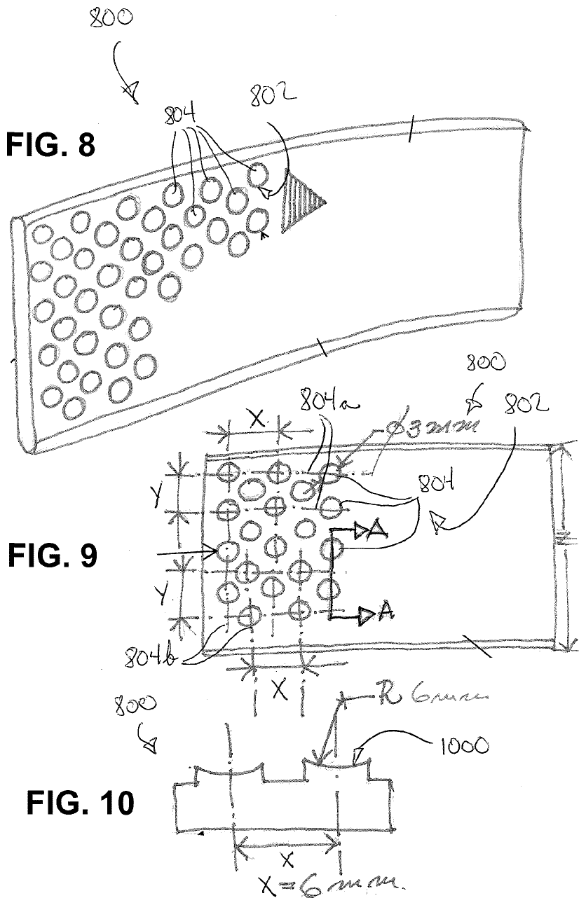

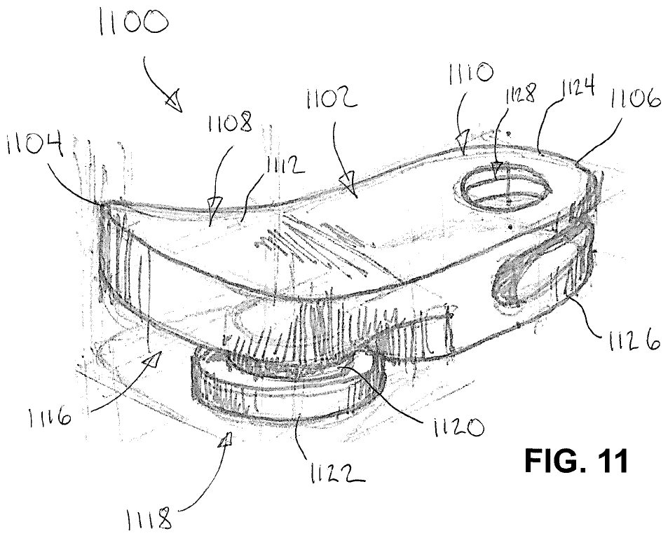

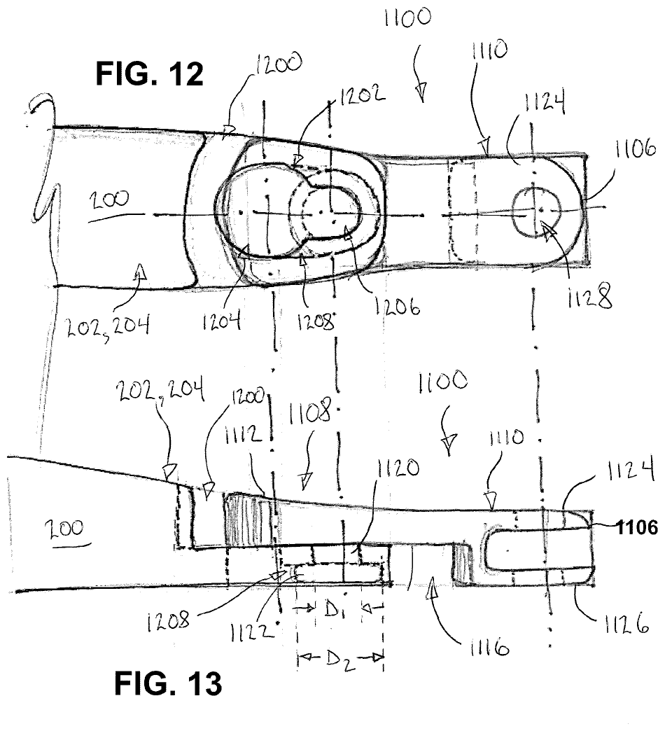

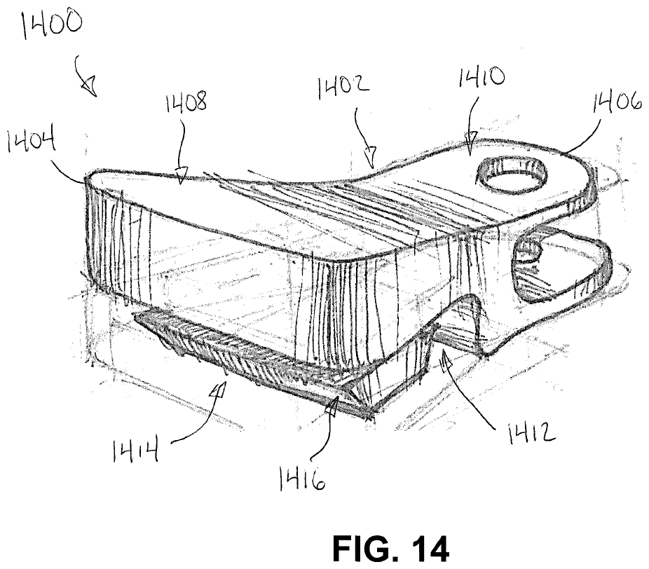

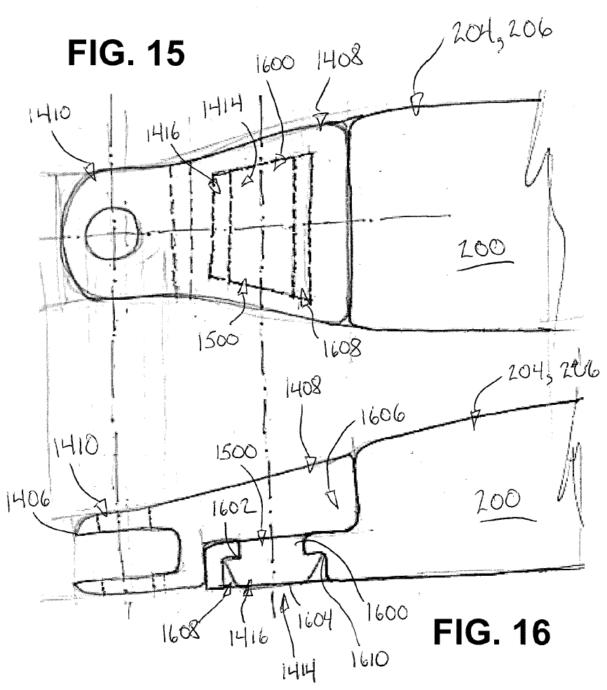

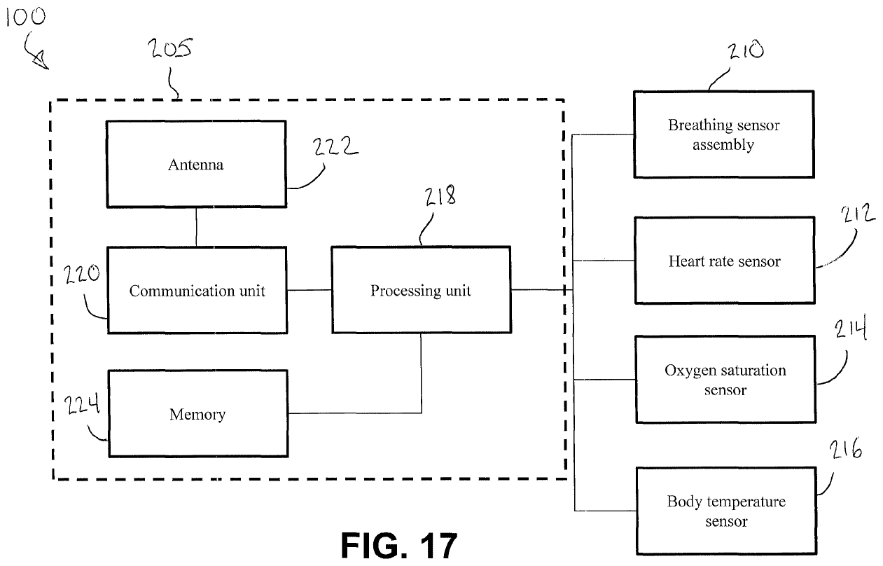

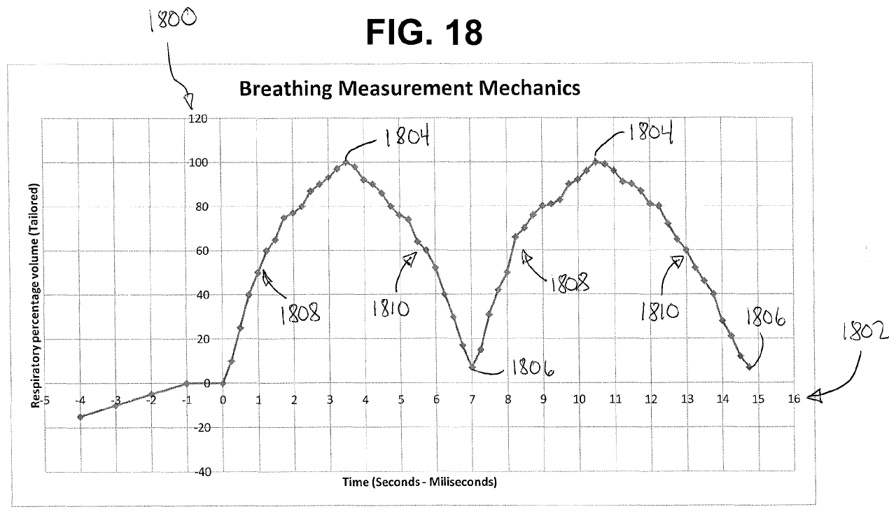

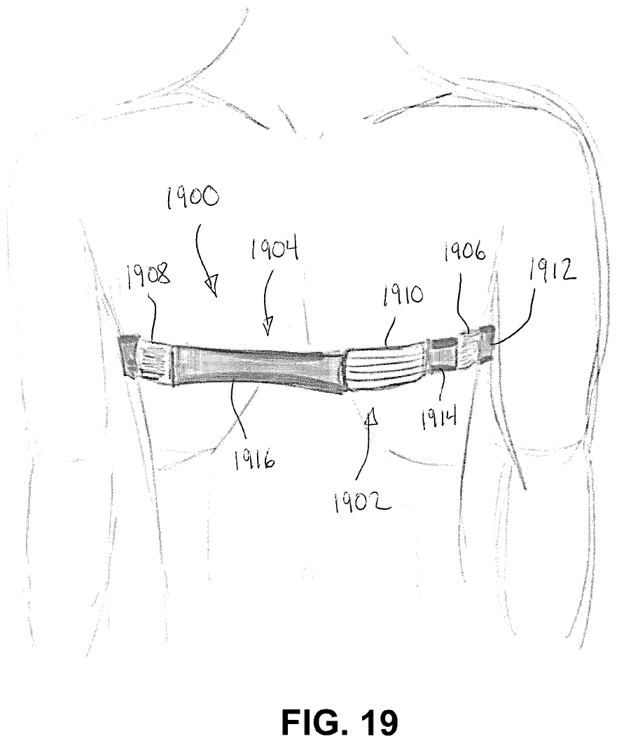

Now turning to FIGS. 5 to 7, the fixation band 102 has a generally constant width along its entire length. Specifically, the fixation band 102 has an upper edge 500 and a lower edge 502 which is generally parallel to the upper edge 500.

The fixation band 102 also has an inner surface 504 adapted to be placed against the user's torso and an outer surface 506 opposite the inner surface 504 adapted to be disposed away from the user's torso. The inner surface 504 of the fixation band 102 is adapted to remain in permanent contact with the user's torso when worn by the user. Specifically, the inner surface 504 is textured to improve friction between the fixation band 102 and the user's skin or upper garment to prevent vertical and/or lateral movement of the fixation band 102 relative to the user's torso. In the illustrated embodiment, the inner surface 504 defines a plurality of adjacent rectangular tread portions 508, each tread portion 508 comprising a plurality of linear protrusions 510 angled relative to the upper and lower edges 500, 502 of the fixation band 102. Specifically, the linear protrusions 510 are angled alternately upwardly in a first tread portion 508a and downwardly in a second tread portion 508b adjacent the first tread portion 508a to form a pattern generally reminiscent of a tire tread pattern.

In one embodiment, the linear protrusions 510 are angled at an angle of about 30 degrees relative to the upper and lower edges 500, 502 of the fixation band 102. In one embodiment, the linear protrusions 510 have a height of about 1 mm and are spaced from each other by a distance of about 2 mm. Furthermore, the linear protrusions 510 have a generally triangular cross-sectional shape with sides angled at an angle of about 45 degrees relative to the outer surface 506 of the fixation band 102, as shown in FIG. 7. Alternatively, the linear protrusions 510 could be angled at a different angle and/or have a different shape and/or cross-sectional shape.

In the illustrated embodiment, the thread portions 508 extend over the entire inner surface 504 of the fixation band 102. Alternatively, only a portion of the inner surface 504 could be textured.

Turning to FIGS. 8 to 10, there is shown a fixation band 800 for the physiological monitoring device 100, in accordance with an alternative embodiment.

In this alternative embodiment, the fixation band 800 has an inner surface 802 which is textured and which comprises a plurality of spaced-apart circular protrusions 804. As shown in FIG. 10, each circular protrusion 804 could have a shape generally reminiscent of a suction cup. Specifically, each circular protrusion 804 has a top surface 1000 which is concave and has a radius of about 6 mm. Still in this alternative embodiment, each circular protrusion 804 has a diameter of about 3 mm. Alternatively, the circular protrusions 804 could have a different diameter. In another embodiment, the top surface 1000 of the circular protrusions 804 could be convex instead of concave. In yet another embodiment, the protrusions 804 may not even be circular and may have another shape such as square, triangular or any other shape that a skilled person would consider appropriate.

As best shown in FIG. 9, the protrusions 804 could be disposed in a staggered pattern to define a plurality of protrusion lines 804a extending in a longitudinal direction relative to the fixation band 800 and a plurality of protrusion columns 804b extending in a transversal direction relative to the fixation band 800. In the alternative embodiment illustrated in FIGS. 8 to 10, the protrusions 804 in each protrusion line 804a are spaced apart uniformly from each other by a predetermined longitudinal distance X and the protrusions 804 in each protrusion column 804b are similarly spaced apart uniformly from each other by a predetermined transversal distance Y. Still in the embodiment illustrated in FIGS. 8 to 10, the predetermined longitudinal distance X is similar to the predetermined transversal distance Y. Specifically, the predetermined longitudinal and transversal distances X and Y are about 6 mm, as measured between the centres of adjacent protrusions 804. Alternatively, the predetermined longitudinal and transversal distances X and Y could be more or less than 6 mm. In yet another embodiment, the predetermined longitudinal distance X could be different from the predetermined transversal distance Y.

Now referring to FIGS. 11 to 13, the device 100 further comprises first and second attachment members 1100 respectively secured to the first and second ends 106, 108 of the fixation band 102. In the illustrated embodiment, the first and second attachment members 1100 are similar to each other. Alternatively, the first and second attachment members 1100 could have different configurations, as will be further explained below.

In the embodiment illustrated in FIGS. 11 to 13, each one of the first and second attachment members 1100 is configured to be removably connected to one of the first and second housing ends 202, 204 of the monitoring assembly 104. Each attachment member 1100 a main body 1102 having a first end 1104 adapted to be removably connected to the monitoring assembly 104 and a second end 1106 adapted to be connected to the fixation band 102.

The main body 1102 is generally divided into a first end portion 1108 located towards the first end 1104 and the second end portion 1110 located towards the second end 1106. The first end portion 1108 comprises an upper face 1112, a lower face 1114 and a recess 1116 defined in the lower face 1114. Specifically, the recess 1116 extends from the first end 1104 towards the second end 1106. In this configuration, the lower face 1114 has a generally stepped profile, with the first end portion 1108 having a generally smaller thickness than the second end portion 1110, as best shown in FIG. 13. In the embodiment illustrated in FIG. 13, the first end portion 1108 further generally tapers from the second end portion 1110 towards the first end 804.

In the embodiment illustrated in FIGS. 11 to 13, the attachment members 1100 further comprises a circular button member 1118 which extends from lower face 1114 at the first end portion 1108 downwardly into the recess 1116. Specifically, the circular button member 1118 comprises a first cylindrical portion 1120 connected to the first end portion 1108 and a second cylindrical portion 1122 connected to the first cylindrical portion 1120 and located away from the first end portion 1108. The first cylindrical portion 1120 has a first diameter D1 and the second cylindrical portion 1122 has a second diameter D2 which is larger than the first diameter D1. The first cylindrical portion 1120 is adapted for engaging a corresponding receiving portion of the monitoring assembly 104 and the second cylindrical portion 1122 is adapted to maintain the first cylindrical portion 1120 in engagement with the corresponding receiving portion of the monitoring assembly 104, as will be further explained below.

Still in the embodiment illustrated in FIGS. 11 to 13, the second end portion 1110 comprises lower and upper connection plates 1124, 1126 which are spaced apart from each other and extend generally parallel to each other to define a yoke-type configuration. Specifically, the lower and upper connection plates 1124, 1126 are adapted to receive a corresponding one of the first and second ends 106, 108 of the fixation band 102. The second end portion 1110 further comprises a fastening hole 1128 extending transversely through the lower and upper connection plates 1124, 1126 for receiving a fastener such as a pin, a rivet, a connecting rod or the like to secure the attachment member 1100 to the fixation band 102.

In the illustrated embodiment, the second end 1106 of the attachment member 1100 is generally convex or rounded to prevent sharp corner from protruding from the sides of the fixation band 102 if the fixation band 102 is not perfectly aligned with the attachment member 1100. Alternatively, the second end 1106 of the attachment member 1100 could simply be straight.

As shown in FIGS. 12 and 13, the first and second housing ends 202, 204 are adapted to receive the attachments members 1100. Specifically, each housing end 202, 204 has a generally stepped profile and comprises a top recess 1200 sized and shaped to receive the first end portion 1108 of one of the attachment members 1100 and a button opening 1202 sized and shaped for receiving the circular button member 1118 of the attachments members 1100.

In the embodiment illustrated in FIGS. 11 to 13, the button opening 1202 is generally pear-shaped and comprises overlapping first and second circular portions 1204, 1206. The first circular portion 1204 is located away from the fixation band 102 and is larger than the second circular portion 1206. Specifically, the first circular portion 1204 is adapted to receive the second cylindrical portion 1122 of the circular button member 1118 and therefore, has a diameter which is equal to or larger than the second diameter D2 of the second cylindrical portion 1122. The second circular portion 1206 is located towards the fixation band 102 and is adapted to receive the first cylindrical portion 1120 of the circular button member 1118. Specifically, the second circular portion 1204 has a diameter which is equal to or larger than the first diameter D1 of the first cylindrical portion 1120, but which is smaller than the second diameter D2 of the second cylindrical portion 1122.

Still in the embodiment illustrated in FIGS. 11 to 13, each housing end 202, 204 further comprises a bottom recess 1208 located below the housing 200, away from the top recess 1200, and in alignment with the button opening 1202. The bottom recess 1208 is generally oblong and has a width which is equal to or greater than the second diameter D2 of the second cylindrical portion 1122.

To assemble the fixation band 102 to the housing 200, the circular button member 1118 is first inserted into the first circular portion 1204 of the button opening 1202, and is lowered until the first end portion 1108 of the attachment member 1100 is received in the top recess 1200. In this configuration, the first cylindrical portion 1120 extends through the first circular portion 1204 and the second cylindrical portion 1122 is now located in the bottom recess 1208. The fixation band 102 can now be pulled away longitudinally from the monitoring assembly 104 such that the second cylindrical portion 1122 slides within the bottom recess 1208 and the first cylindrical portion 1120 slides from the first circular portion 1204 into the second circular portion 1206. In this configuration, the attachments member 1100 is prevented from being separated from the corresponding housing end 202, 204 because the second cylindrical portion 1122 has a diameter D2 which is greater than the width of the bottom recess 1208 and therefore cannot pass through the second circular portion 1206. To separate the attachment member 1100 from the housing 200, the attachment member 1100 is pushed towards the housing 200 until the second cylindrical portion 1122 is again in alignment with the first circular portion 1204.

In one embodiment, the fixation band 102 and the housing 200 are configured to be always tensioned around the user's torso, such that the first cylindrical portion 1120 is always urged towards the second circular portion 1206 when the device 100 is worn by the user. In this embodiment, the fixation band 102 and/or the housing 200 could be slightly elastic to create this tension.

In another embodiment, the fixation band 102 could further comprise an adjustment device such as an adjustment buckle which allows the fixation band 102 to be shortened and therefore tightened around the user's torso. In this embodiment, the device 100 is fastened around the user's torso by attaching the attachment members 1100 to the first and second housing ends 202, 204 and by further tightening the fixation band 102 to prevent the first cylindrical portion 1120 of the circular button member 1118 from sliding back in alignment with the first circular portion 1204 of the housing 200.

Now turning to FIGS. 14 to 16, there is shown an attachment member 1400, in accordance with an alternative embodiment. The attachment member 1400 comprises a main body 1402 having a first end 1404 adapted to be securely connected to the monitoring assembly 104 and a second end 1106 adapted to be connected to the fixation band 102. The main body 1402 is generally divided into a first end portion 1408 located towards the first end 1404 and the second end portion 1410 located towards the second end 1412.

In the embodiment illustrated in FIGS. 14 to 16, the second end portion 1410 is generally similar to the second end portion 1110 of the attachment member 1100. Specifically, the second end portion 1410 of the attachment member 1400 is adapted to be secured to a corresponding one of the first and second ends 106, 108 of the fixation band 102.

The first end portion 1408 further defines a recess 1412, which is generally similar to the recess 1116 of the attachment member 1100, and a hook member 1414 which extends downwardly from the recess 1412. The hook member 1414 comprises a tip portion 1416 which has a cross-section generally shaped like a truncated arrowhead and a generally rectangular connecting portion 1600, best shown in FIG. 16, connecting the tip portion 1416 to the first end portion 1408. As shown in FIG. 16, the tip portion 1416 tapers from a base portion 1602 which is wider than the connecting portion 1600 and an end portion 1604 which is narrower than the base portion 1602.

As shown in FIGS. 15 and 16, the first and second housing ends 202, 204 are adapted to receive the attachments members 1400. Specifically, each housing end 202, 204 has a generally stepped profile and comprises a top recess 1606 sized and shaped to receive the first end portion 1408 of one of the attachment members 1400 and a hook opening 1500 sized and shaped for receiving the hook member 1414 of the attachments members 1400.

In the embodiment illustrated in FIGS. 15 and 16, both the hook member 1414 and the hook opening 1500 have corresponding trapezoidal shapes when viewed in a top view. Specifically, the hook member 1414 is slightly smaller than the hook opening 1500 to be able to be received in the hook opening 1500. Each housing end 202, 204 of the housing 200 further comprises a bottom recess 1608 located below the housing 200, away from the top recess 1602, and in alignment with the hook opening 1500. The hook opening 1500 is sized and shaped to receive the connecting portion 1600 and the bottom recess 1608 is sized and shaped to receive the base portion 1602 of the hook member 1414. The bottom recess 1608 is therefore generally wider than the hook opening 1500 and thereby defines an internal shoulder 1610 inside the bottom recess 1608 between the bottom recess 1608 and the hook opening 1500.

In one embodiment, at least one of the first or second housing end 202, 204 and the tip portion 1416 of the hook member 1414 is at least slightly deformable to allow the hook member 1414 to be inserted into the hook opening 1500 until the tip portion 1416 is received in the bottom recess 1608. Specifically, the base portion 1602, which is wider than the hook opening 1500, is able to bend slightly as the tip portion 1416 passes through the hook opening 1500. Once the tip portion 1416 is inserted past the hook opening 1500 such that the first end portion 1408 of the attachment member 1400 is received in the top recess 1606, the base portion 1602 abuts the internal shoulder 1610 inside the bottom recess 1608 and prevents the tip portion 1416 from being removed from the bottom recess 1608. In one embodiment, the attachment member 1400 is permanently attached to the housing 200. Alternatively, the hook member 1414 could be removable from the hook opening 1500 by forcefully pulling the attachment member 1400 away from the corresponding housing end 202, 204.

In one embodiment, the device 100 comprises the attachment member 1100 illustrated in FIGS. 11 to 13 to be fastened to one of the first and second housing ends 202, 204 and the attachment member 1400 illustrated in FIGS. 14 to 16 to be fastened to the other one of the first and second housing ends 202, 204. This allows the fixation band 102 to be permanently secured to the housing 200 via one attachment member 1400 while still be detachable and attachable to the housing 200 via the other attachment member 1100 to allow the device 100 to be fastened around the user's torso. Alternatively, the device 100 could comprise attachment members which are both similar to the attachment member 1100 illustrated in FIGS. 11 to 13 or to attachment member 1400 illustrated in FIGS. 14 to 16. In yet another embodiment, the attachment members could be configured differently.

To use the device 100 described above, the device 100 is first fastened around the user's torso. As described above, the device 100 could comprise the attachment member 1100 and the attachment member 1400. In this embodiment, the device 100 is provided with the first end 106 of the fixation band 102 secured to the second end portion 1110 of the attachment member 1100 and with the second end 108 of the fixation band 102 secured to the second end portion 1410 of the attachment member 1400. Furthermore, the device 100 may also be provided with the first end portion 1408 of the attachment member 1400 attached to a corresponding one of the first and second housing ends 202, 204, as described above.

To fasten the device 100 around the user's torso, the device 100 is first looped around the user's torso, generally in the intercostal valley defined between the fifth and sixth true ribs of the user, and the first end portion 1108 of the attachment member 1100 is attached to the other one of the first and second housing ends 202, 204 as described above.

In one embodiment, the device 100 is disposed such that the housing 200 is positioned generally on a left side of the user's torso, relative to the body's sagittal plane. Specifically, the device 100 may be disposed such that the housing 200 is positioned generally in alignment with the user's heart. This configuration may improve the precision of measurements from the heart rate sensor 212 and the oxygen saturation sensor 214, as a skilled person will appreciate.

The device 100, once fastened around the user's torso, could then be used to determine a respiratory rate of the user. In one embodiment, the device 100 may first be activated using the power switch. The device 100 could further be calibrated to the user. Specifically, the user first exhales completely such that the user's torso is at a minimum volume and strain is measured from the strain gauge to determine a minimum strain value. Specifically, the minimum strain value could correspond to a minimum voltage value measured from the strain gauge 402. The user could then inhale completely such that the user's torso is at a maximum volume and strain is measured from the strain gauge 402 to determine a maximum strain value. Specifically, the maximum strain value could correspond to a maximum voltage value measured from the strain gauge 402. Alternatively, the maximum strain value could be determined before the minimum strain value.

In yet another embodiment, the user could simply breathe deeply, thereby successively inhaling and exhaling for a certain period of time. The minimum and maximum voltage values could then be determined, and the minimum and maximum strain value could also be determined, with the minimum strain value corresponding to the minimum voltage value and the maximum strain value corresponding to the maximum voltage value.

Now turning to FIG. 18, the device 100 can then be used to monitor the respiratory rate of the user over a period of time. Specifically, strain value is measured at a predetermined frequency over a period of time to determine a pattern of successive inhalations and exhalations of the user. For example, the strain value may be measured at a frequency of four measurements per second. Alternatively, the strain value may be measured at a different frequency.

In the embodiment illustrated in FIG. 18, the respiratory rate of the user is represented as a value of respiratory percentage volume 1800 as a function of time 1802. It will be understood that the respiratory percentage volume generally represents an amount of air inside the user's lungs relative to the user's total lung capacity. Specifically, the measured strain value is scaled such that the maximum strain value corresponds to a maximum respiratory percentage volume 1804 of about 100% and the minimum strain value corresponds to a minimum respiratory percentage volume 1806 of about 0%. In this representation, an increase 1808 in the measured voltage value corresponds to an inhalation by the user and a decrease 1810 in voltage value corresponds to an exhalation by the user.

In one embodiment, the value of respiratory percentage volume as a function of time may be plotted and displayed on a display of an external terminal in communication with the controller 205 via the communication unit 220. This representation may be updated in real time as additional strain values are measured. Alternatively, the strain values may be stored in the memory 224 or in an external memory and be plotted in a line chart representation in response to a request from the user.

In one embodiment, in addition to a representation of the value of respiratory percentage volume as a function of time, the controller 205 may be adapted to calculate a current respiratory rate value and provide the current respiratory rate value to the user. For example, the controller 205 may be adapted to calculate a number of maximum and/or minimum maximum respiratory percentage volumes measured in a certain period of time, corresponding to a number of inhalations and/or exhalations in this period of time. The calculated number of maximum and/or minimum maximum respiratory percentage volumes can then be divided by the certain period of time to obtain the current respiratory rate value. The controller 205 may be adapted to calculate the current respiratory rate value every 5 seconds, for example, and the certain period of time could be a 60-second period prior to each calculation. Alternatively, the controller 205 may be adapted to calculate the current respiratory rate value at a different frequency and/or over a different period of time.

The controller 100 could further be adapted to provide an indication to the user when the calculated current respiratory rate value is above or below a predetermined threshold. The predetermined threshold could be selectable by the user, and/or could be based on at least one parameter such as the user's age and weight, for example. In one embodiment, the indication could be a visual indication and be provided by the indicator LED as explained above. Alternatively, the indication could be a tactile indication and be provided by the haptic vibration actuator as explained above. In yet another embodiment, the indication could be an audio indication provided by a speaker operatively connected to the controller or by headphones operatively connected wirelessly or via a wire to the controller 205.

In one embodiment, the heart rate, the blood oxygen saturation and the body temperature of the user are measured using the heart rate sensor 212, the oxygen saturation sensor 214 and the body temperature sensor 216, respectively. The heart rate, the blood oxygen saturation and the body temperature of the user could be measured at a predetermined frequency. Alternatively, the heart rate, the blood oxygen saturation and the body temperature of the user could be measured generally similarly to the respiratory rate. Specifically, the heart rate, the blood oxygen saturation and the body temperature could be measured at predetermined frequencies over certain periods of time and be averaged over the certain periods of time. As with the respiratory rate, the controller 205 could be adapted to provide an indication to the user when the measured heart rate, blood oxygen saturation and/or body temperature is above or below a predetermined threshold. The indication could comprise at least one of a visual indication, a tactile indication and an audio indication. Alternatively, the indication could comprise any other indication that a skilled person would consider to be appropriate.

In one embodiment, the measurements of respiratory rate, heart rate, blood oxygen saturation and body temperature are stored in the memory 224. Alternatively, the measurements could also be stored in an external memory of an external terminal operatively connected to the controller 205 wirelessly or through a wired connection. It will be appreciated that this would allow the measurements to be further plotted and/or further analyzed by the user using an external analysis software which could be provided on the external terminal.

It will be appreciated that the device 100 is relatively lightweight, compact and comfortable, and is therefore particularly well-adapted to monitor physiological parameters during a physical activity such as walking, running, cycling, working out and the like. The device 100 could be used to monitor physiological parameters during a particular activity session, for example, and provide an indication such as an alarm signal when the measured values are above or below predetermined thresholds to prevent certain undesirable conditions such as heatstroke and extreme exhaustion.

The device 100 could also be used to monitor physiological parameters over a relatively long period of time. For example, the measurements taken during a first activity session may be analyzed using an analysis software to allow the activities performed during the activity session to be adjusted for a second activity session according to a physiological response of the user to the first activity session. This may be useful for improving recovery time after an activity session and/or to maximize long time performance during the activity session. The device 100 could also simply be used to track progress of the user during a training program comprising multiple training sessions distributed over a few weeks or a few months.

In one embodiment, the device 100 could also be used to monitor physiological parameters during normal daily activities instead of specific physical activity sessions, either for medical purposes or simply to allow the user to evaluate the amount of physical activity performed during a normal day.

It will be appreciated that the configuration described above is merely provided as an example, and that the physiological monitoring device could instead be configured according to one of various alternative configurations.

For example, FIG. 19 shows a physiological monitoring device 1900 in accordance with an alternative embodiment. Similarly to the device 100 illustrated in FIG. 1, the device 1900 comprises a monitoring assembly 1902 and a fixation band 1904 connected to the monitoring assembly and adapted to maintain the monitoring assembly 1902 against the user's torso. In this embodiment, the monitoring assembly 1902 comprises left and right breathing sensor subassemblies 1906, 1908 and a main subassembly 1910 located between the left and right breathing sensor subassemblies 1906, 1908. Still in this embodiment, the fixation band 1904 comprises a rear fixation band portion 1912 extending between the left and right breathing sensor subassemblies 1906, 1908 and left and right front fixation band portions 1914, 1916 extending respectively between the left breathing sensor subassembly 1906 and the main subassembly 1910 and between the right breathing sensor subassembly 1908 and the main subassembly 1910.

When the device 1900 is worn by the user, the rear fixation band portion 1912 is disposed at the back of the user's torso, and the main subassembly 1910 and the left and right front fixation band portions 1914, 1916 are disposed generally at the front of the user's torso. Specifically, the left front fixation band portion 1914 is slightly longer than the right front fixation band portion 1916 such that the main subassembly 1910 may be positioned over the user's heart and the left and right breathing sensor subassemblies 1906, 1908 may disposed generally symmetrically on either side of the user's sagittal plane.

In this embodiment, each breathing sensor subassembly 1906, 1908 is generally similar to the breathing sensor assembly 210 illustrated in FIGS. 1 to 17 and the main subassembly 1910 contains all of the remaining elements of the monitoring assembly 104 illustrated in FIGS. 1 to 17. Still in this embodiment, the strain value may be measured by combining the voltage values from both breathing sensor subassemblies 1906, 1908. It will be appreciated that the use of two deformation sensors instead of a single deformation sensor may contribute to providing a more precise deformation measurement and therefore a more precise determination of the user's breathing rate. It will also be understood that in another embodiment, the device could comprise more than two deformation sensors.

While illustrative and presently preferred embodiment(s) of the invention have been described in detail hereinabove, it is to be understood that the inventive concepts may be otherwise variously embodied and employed and that the appended claims are intended to be construed to comprise such variations except insofar as limited by the prior art.

1. A physiological monitoring device comprising:
a fixation band adapted to be fastened around a user's torso;
a monitoring assembly fastened to the fixation band, the monitoring assembly comprising:
at least one deformable member adapted to be deformed in response to an expansion of the user's torso;
at least one deformation sensor operatively connected to the at least one deformable member for measuring a deformation of the deformable member; and
a processing unit operatively connected to the at least one sensor for determining a physiological parameter value of the user based on the measured deformation.

2. The device as claimed in claim 1, wherein the physiological parameter value comprises a respiratory rate of the user.

3. The device as claimed in claim 1, wherein the deformable member comprises a deformable substrate plate.

4. The device as claimed in claim 1, wherein each deformation sensor comprises a strain gauge.

5. The device as claimed in claim 1, wherein the monitoring assembly further comprises a heart rate sensor.

6. The device as claimed in claim 1, wherein the monitoring assembly further comprises an oxygen saturation sensor.

7. The device as claimed in claim 1, wherein the monitoring assembly further comprises a body temperature sensor.

8. The device as claimed in claim 1, wherein the monitoring assembly further comprises a housing adapted to house the at least one deformable member, the at least one deformation sensor and the processing unit.

9. The device as claimed in claim 1, wherein the monitoring assembly further comprises a battery operatively connected to the processing unit.

10. The device as claimed in claim 1, wherein the monitoring assembly further comprises a communication unit operatively connected to the processing unit for allowing data to be exchanged between the monitoring assembly and at least one external terminal.

11. The device as claimed in claim 1, wherein the monitoring assembly further comprises a memory for storing data received by the processing unit from the at least one sensor.

12. The device as claimed in claim 1, wherein the fixation band comprises an outer surface adapted to be disposed away from the user's torso and an inner surface adapted to be disposed towards the user's torso, the inner surface being textured to improve friction between the fixation band and the user's skin.

13. The device as claimed in claim 3, wherein the deformable substrate plate is made from polycarbonate.

14. The device as claimed in claim 4, wherein the deformable substrate plate has a thickness of about 2 mm.

15. The device as claimed in claim 4, wherein the deformable substrate plate is generally rectangular.

16. The device as claimed in claim 6, wherein the deformable substrate plate comprises four anchoring holes disposed at the four corners of the deformable substrate plate.

17. The device as claimed in claim 6, wherein each one of the at least one deformation sensor is disposed at the center of a corresponding one of the at least one deformable substrate plate.

18. The device as claimed in claim 8, wherein the deformation sensor is disposed and oriented on the corresponding deformable substrate plate so as to allow measurements of strain longitudinally along the fixation band and the monitoring assembly.

19. The device as claimed in claim 10, wherein each strain gauge comprises a foil strain gauge.

20. The device as claimed in claim 12, wherein the heart rate sensor comprises a green light emitting diode and a corresponding photoreceptor.

21. The device as claimed in claim 14, wherein the oxygen saturation sensor comprises an infrared light emitting diode and a corresponding photoreceptor.

22. The device as claimed in claim 17, wherein the housing is overmolded over the at least one deformable member, the at least one deformation sensor and the processing unit.

23. The device as claimed in claim 19, wherein the battery is rechargeable.

24. The device as claimed in claim 21, wherein the at least one external terminal comprises at least one of a smart phone, a smart watch and a personal computer.

25. The device as claimed in claim 21, wherein the monitoring assembly further comprises an antenna operatively connected to the communication unit for enabling data to be transmitted wirelessly by the communication unit.

26. A method for measuring a respiratory rate of a user, the method comprising:
a) providing a monitoring assembly comprising at least one deformable member adapted to be deformed in response to an expansion of the user's torso and at least one deformation sensor operatively connected to the at least one deformable member for measuring a deformation of the deformable member;
b) placing the monitoring assembly against a user's torso using a fixation band attached to the monitoring assembly;
c) measuring a strain value generated by the at least one deformation sensor in response to a plurality of inhalations and exhalations of the user over a predetermined period of time.

27. The method as claimed in claim 26, wherein measuring a strain value comprises:
a) measuring an increase in strain value from the at least one deformation sensor, the increase being indicative of an inhalation by the user;
b) measuring a decrease in strain value from the at least one deformation sensor, the decrease being indicative of an exhalation by the user.
